(19) United States
(12) Patent Application Publication
(10) Pub. No.: US 2018/0193464 A1
(43) Pub. Date: Jul. 12, 2018

(54) COMPOSITIONS AND METHODS FOR INJECTION OF A BIODEGRADABLE POLYMER-BASED DELIVERY SYSTEM

(71) Applicant: Heron Therapeutics, Inc., Redwood City, CA (US)

(72) Inventor: John Whelan, Cupertino, CA (US)

(21) Appl. No.: 15/727,543

(22) Filed: Oct. 6, 2017

Related U.S. Application Data
(63) Continuation of application No. 14/264,489, filed on Apr. 29, 2014, now abandoned.
(60) Provisional application No. 61/817,748, filed on Apr. 30, 2013.

(51) Int. Cl. A61K 47/34; A61K 9/00; A61K 31/439

(52) U.S. Cl. CPC A61K 47/34; A61K 9/0019; A61K 9/0004; A61K 31/439

(57) ABSTRACT

A method for administering a polymeric-based delivery system to a subject is described. The method comprises contacting a housing comprising a polymer-based delivery system with a heat-generating component for a period of time; and injecting the delivery system into the subject.

### CROSS-REFERENCE TO RELATED APPLICATIONS

This application is a continuation of U.S. application Ser. No. 14/264,489, filed Apr. 29, 2014, which claims the benefit of U.S. Provisional Application No. 61/817,748, filed Apr. 30, 2013, the disclosures of which are incorporated herein by reference in their entirety.

### TECHNICAL FIELD

The present disclosure is directed to methods and systems for delivery of a biodegradable polyorthoester polymer, an excipient, and a drug to a patient.

### BACKGROUND

Development of polymer-based depot systems for parenteral controlled release of drugs has progressed significantly in recent decades and these systems have proven to provide an effective and efficient means of drug delivery. Such drug delivery systems provide easy access to systemic circulation with rapid drug absorption while providing extended exposure to the drug. Other advantages include ease of application, localized delivery for a site-specific action in the body, reduced dosing frequency and increased dosing compliance.

Many factors influence the design and performance of such systems, such as the physical/chemical properties of the drug, the physical/chemical characteristics of the system's components and the performance/behavior relative to other system components once combined, external/environmental conditions at the site of application. In designing polymer based systems for delivery of a drug, the desired rate of drug delivery and onset, the drug delivery profile, and the intended duration of delivery all must be considered.

There remains a need for polymer-based compositions that offer the flexibility to modulate or tailor the rate of drug release. The present compositions satisfy this need.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method is provided for administering to a subject a semi-solid therapeutic substance comprised of a biodegradable polyorthoester which is contained within a housing or device which is applicable for storage and/or administration of the substance, and contacting the housing or device with a heat source for a period of time to affect the fluid characteristics of the substance.

In one embodiment, the period of time for the heat source to reach operating temperature ranges from about 1 min (minutes) to 25 min, and preferably in the range from 2 min to 10 min.

In one embodiment, the housing or device achieves a temperature in the range of about 20° C. to 50° C. and preferably a temperature in the range of 30° C. to 40° C.

In one embodiment, the method comprises administering the substance using the device by injecting subcutaneously, intradermally or intramuscularly.

In one embodiment, the device contains an injection needle with a size in the range of 14 gauge to 25 gauge, preferably in the range of 16 to 20 gauge.

In one embodiment, the injection is performed within a range of 5 sec (seconds) to 2 min, preferably in the range of 15 sec to 1 min.

In one embodiment, the viscosity of the substance decreases as the temperature of the polymer increases. In one embodiment the substance decreases to 40,000 cP (centipoise) at body temperature, 37° C.

In one embodiment, the housing or device and the substance are heated through direct contact to the heating source.

In one embodiment, the administration is performed after separating the heat source from the housing or device. In still another embodiment, the administering is done while the heat source is still in contact with the housing or device.

In one embodiment, the administered substance comprises an excipient which is readily miscible with the polyorthoester. In another embodiment, the administered substance comprises at least one active agent which is admixed with the polyorthoester.

In one embodiment, the excipient is a pharmaceutically acceptable, polyorthoester-compatible liquid excipient selected from polyethylene glycol ether derivatives having a molecular weight between about 200 Da (Daltons) and 4000 Da, polyethylene glycol copolymers having a molecular weight between about 400 Da and 4000 Da, mono-, di-, or tri-glycerides of a C₂₋₁₉ aliphatic carboxylic acid or a mixture of such acids, alkoxylated tetrahydrofurfuryl alcohols and their C₁₋₄ alkyl ethers and C₂₋₁₉ aliphatic carboxylic acid esters, and biocompatible oils.

In one embodiment, the excipient is an organic solvent having a water solubility of greater than 25% by weight at room temperature where room temperature is defined as being in the range of 18° C. to 27° C. In another embodiment, the solvent is a dipolar aprotic solvent.

In one embodiment, the substance has a viscosity at room temperature which varies from about 30,000 cP to 250,000 cP where room temperature is defined as being in the range of 18° C. to 27° C.

In one embodiment, the active agent is present in an amount between about 1 to 10 percent by weight or about 1% to 5% by weight of the substance.

In one embodiment, the excipient is present in an amount between about 10 to 35 percent or about 10% to 20% by weight of the substance.

In one embodiment, the heat source comprises an exothermic thermochemical composition.

In one embodiment, the exothermic thermochemical composition is a liquid solution. In another embodiment, the exothermic composition is a supercoolable salt solution. In yet another embodiment, the exothermic composition is an aqueous supersaturated sodium acetate solution. In one embodiment comprising a supersaturated sodium acetate solution, the exothermic reaction is initiated by a physical trigger which provides an enucleation source for crystallization of the solution.

In one embodiment, the heat source comprises a solid exothermic thermochemical composition. In another embodiment, the exothermic thermochemical composition is an iron powder. In another embodiment the exothermic reaction is initiated by exposure of iron powder to air.

In one embodiment, the heat source comprises an enclosure which encases the exothermic thermochemical composition and which comprises a first surface and a second surface. In another embodiment the first and second surfaces of the enclosure are comprised of a flexible polymer. In another embodiment, the first and/or the second surface comprises an insulating material. In another embodiment, the enclosure comprises a third surface or layer comprising an insulating layer which is bonded to the first or the second surface on three edges of the enclosure creating a pocket between the insulating layer and the surface within which the delivery system containing the substance can be placed in order to apply heat.

In one embodiment, the first or the second surface is permeable to air. In another embodiment, the heat source further comprises a third surface bonded to and releasable from the permeable surface wherein removal of the third surface allows air to enter the enclosure and initiate the exothermic thermochemical reaction.

In one embodiment, the heat source enclosure contains a sleeve or tubular element having a lumen sized to accept the housing or device containing the therapeutic substance. In one embodiment the sleeve in disposed between the surfaces of the enclosure and is open on one or both ends to accept the housing or device. In another embodiment, the sleeve is bonded to or formed on the top of one of the enclosure surfaces.

In one embodiment, the heat source enclosure is designed to fold over itself creating a space within which the housing or device containing the substance can be placed for warming. In another embodiment, the enclosure further comprises a length of adhesive tape, a hook and loop fastener or other means to secure the enclosure in the folded position.

In one embodiment, the exothermic thermochemical composition comprises two or more chemical components which are physically separated by a barrier, wherein disruption of the barrier results in the combination and the separate chemical components, wherein the combination results in activation and heat production.

In one aspect a kit is provided, wherein the kit comprises a housing or device in which the biodegradable polymeric substance is contained, and a heat source, wherein the substance comprises a polyorthoester, an excipient, and an active agent.

In one embodiment, the device comprises a syringe. In another embodiment, the housing is a vial suitable for storage of the substance which further comprises means to allow the transfer of the substance to an injection device such as a syringe.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. Definitions

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

“Semi-solid” denotes the mechano-physical state of a material that is flowable under moderate stress. More specifically, the semi-solid material should have a viscosity between about 10,000 cP and 3,000,000 cP, especially between about 50,000 cP and 500,000 cP. Preferably the formulation is easily syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

“Bioerodible,” “bioerodibility” and “biodegradable,” which are used interchangeably herein, refer to the degradation, disassembly or digestion of a polymer by action of a biological environment, including the action of living organisms and most notably at physiological pH and temperature. As an example, a principal mechanism for bioerosion of a polyorthoester is hydrolysis of linkages between and within the units of the polyorthoester.

As used herein, the term “emesis” includes nausea and vomiting.

Solubility values of solvent in water are considered to be determined at 20° C.

“Molecular mass” in the context of a polymer such as a polyorthoester, refers to the nominal average molecular mass of a polymer, typically determined by size exclusion chromatography, light scattering techniques, or velocity. Molecular weight can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 3.0, less than about 2.75, less than about 2.25, less than about 1.5, and less than about 1.03.

A “polymer susceptible to hydrolysis” and “polyorthoester” refers to a polymer that is capable of degradation, disassembly or digestion through reaction with water molecules. Such a polymer contains hydrolyzable groups in the polymer. Examples of polymers susceptible to hydrolysis may include, but is not limited to, polymers described herein, and those described in U.S. Pat. Nos. 4,079,038, 4,093,709, 4,131,648, 4,138,344, 4,180,646, 4,304,767, 4,957,998, 4,946,931, 5,968,543, 6,613,335, and 8,252,304, U.S. Patent Publication No. 2007/0265329, and U.S. Provisional Patent Application No. 61/789,469, filed on Mar. 15, 2013, each of which is incorporated by reference in its entirety.

“Polyorthoester-compatible” refers to, in one particular aspect of the properties of the polyorthoester, the properties of an excipient which, when mixed with the polyorthoester, forms a single phase and does not cause any physical or chemical changes to the polyorthoester.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

As used herein, the term “transparent”, when used in the context of material, refers to the property of permitting viewing of contents beyond the opposing side of such material in a substantially clear manner. The term is meant to include colored transparent materials.

As used herein, the term “trigger” when used in the context of a crystallization activator is meant to refer to the generally planar and flexible devices that, upon flexing within thermochemical fluid, initiate crystallization and the subsequent exothermic effect. Such devices are described, for example, in U.S. Pat. Nos. 4,460,546, 4,572,158, 4,872,442, 5,143,048, 5,736,110, and 6,283,116, the entire texts of which are incorporated herein by reference.

### II. Delivery System and Composition

In one aspect, a method and a system for administering a substance comprising a polymeric-based drug delivery system substance to a subject is provided. As will be described below, the method comprises contacting the housing or device in which the substance is contained with a heat source for a period of time. In one embodiment, the heat source is a self-generating heat source. In another embodiment, the heat source is contacted, directly or indirectly, with the polymeric-based substance. Described below are the polymeric-based drug delivery system substance and its components and exemplary heat sources.

The polymeric-based systems and compositions described herein comprise a biodegradable polyorthoester polymer combined with a biocompatible organic solvent as an excipient, and find use, for example, as drug delivery systems or as medical or surgical devices. The excipient in the system may be used to modulate both the release profile of an active agent from the system as well as the viscosity of the system, and the response of the system to the heat source. In some embodiments, the as formulated delivery system which provides an optimal release profile may have a viscosity which is not conducive with administration to the subject. For example, a higher viscosity delivery system will require a larger needle, resulting in increased discomfort for patient and possibly decreased compliance by the patient. The method herein contemplates altering the excipient, excipient amount, and the responsiveness of the delivery system to the heat source in order to reduce the viscosity of the delivery system.

Accordingly, described below are compositions and methods for the preparation of a delivery system containing an active agent, wherein the delivery system has a viscosity which allows for the use of a small needle (e.g., about 18-26 gauge). Delivery systems having a relatively high viscosity can be warmed immediately prior to injection in order to reduce the viscosity of the injected material so that it can be easily injected into the body with standard syringes and small gauge needles.

### A. Polyorthoester Polymers

In one embodiment, the compositions and delivery systems described herein are comprised of a polyorthoester of formula I, formula II, formula III or formula IV:

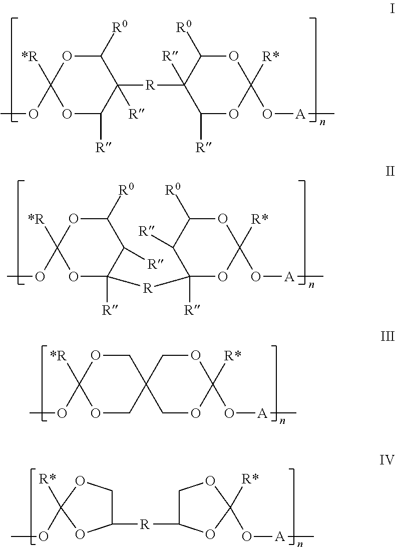

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

Rº, R″ and R′″ are each independently H or C₁₋₄ alkyl;

n is an integer of at least 5; and

A is a diol.

In another embodiment, the compositions and delivery systems described herein are comprised of a polyorthoester of formula I, formula II, formula III or formula IV:

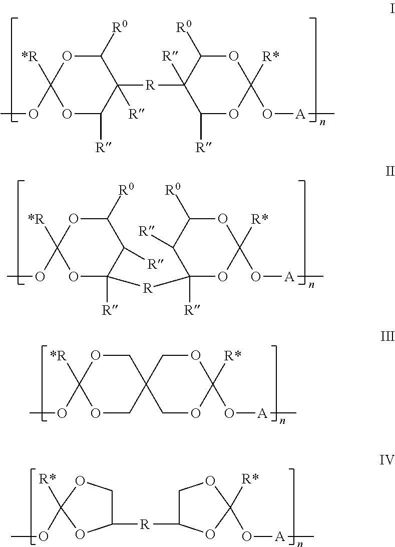

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

Rº, R″ and R′″ are each independently H or C₁₋₄ alkyl;

n is an integer of at least 5; and

A is R′, R², R³, or R⁴, where

R¹ is:

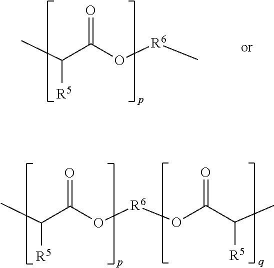

where:
p and q are integers that vary from between about 1 to 20 and the average number of p or the average of the sum of p and q is between 1 and 7 in an least a portion of the monomeric units of the polymer;

R⁵ is hydrogen or C₁₋₄ alkyl; and

R⁶ is:

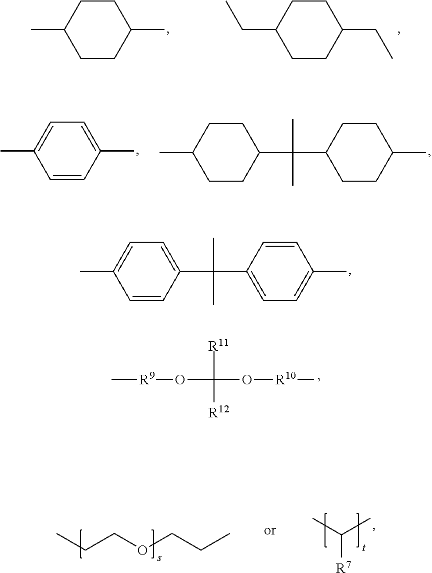

where:

s is an integer of 0 to 30;

t is an integer of 2 to 200; and

R⁷ is hydrogen or C₁₋₄ alkyl;

R² is:

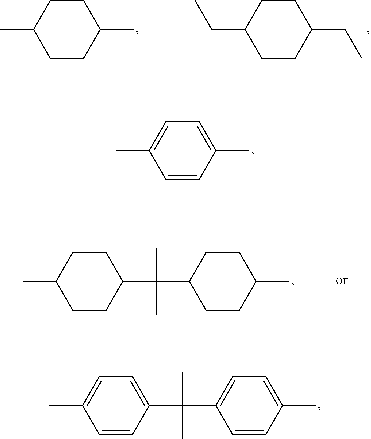

R³ is:

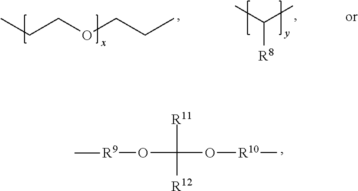

where:

x is an integer of 0 to 100;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene; and

R⁴ is the residue of a diol containing at least one functional group independently selected from amide, imide, urea, and urethane groups.

In some embodiments, A is R¹, R³, or R⁴, where

R¹ is:

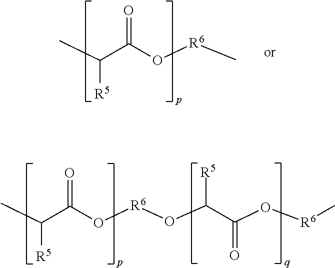

where:
p and q are integers that vary from between about 1 to 20 and the average number of p or the average of the sum of p and q is between 1 and 7 in an least a portion of the monomeric units of the polymer;

R³ and R⁶ are each independently:

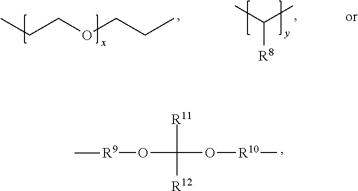

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene;

R⁴ is a residual of a diol containing at least one functional group independently selected from amide, imide, urea and urethane groups; and R⁵ is hydrogen or C₁₋₄ alkyl.

In some embodiments, the concentration of the polyorthoester ranges from 1% to 99% by weight. In other embodiments, the polyorthoester has a molecular weight between 3,000 and 10,000. In another embodiment, the fraction of the A units that are of the formula R¹ is between 5 and 15 mole percent.

In another embodiment, the polyorthoester is of formula I, where: none of the units have A equal to R²;

R³ is:

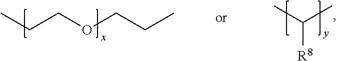

where:

x is an integer of 0 to 10;

y is an integer of 2 to 30; and

R⁶ is:

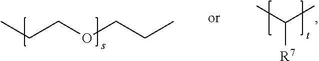

where:

s is an integer of 0 to 10;

t is an integer of 2 to 30; and

R⁵, R⁷, and R⁸ are independently hydrogen or methyl.

In another embodiment, R³ and R⁶ are both —(CH₂—CH₂—O)₂—(CH₂—CH₂)—; R⁵ is methyl; and p is 1 or 2. In another embodiment, R³ and R⁶ are both —(CH₂—CH₂—O)₉—(CH₂—CH₂)—; R⁵ is methyl; and p or the sum of p and q is on average 2. In another variation, the polyorthoester is of formula I, R is —(CH₂)_{b}—O—(CH₂)_{c}—; where b and c are both 2; R* is a C₂ alkyl.

The polyorthoester, as shown in formula I, formula II, formula III and formula IV, in some embodiments, is one of alternating residues of a diketene acetal and a diol, with each adjacent pair of diketene acetal residues being separated by the residue of one polyol, such as a diol.

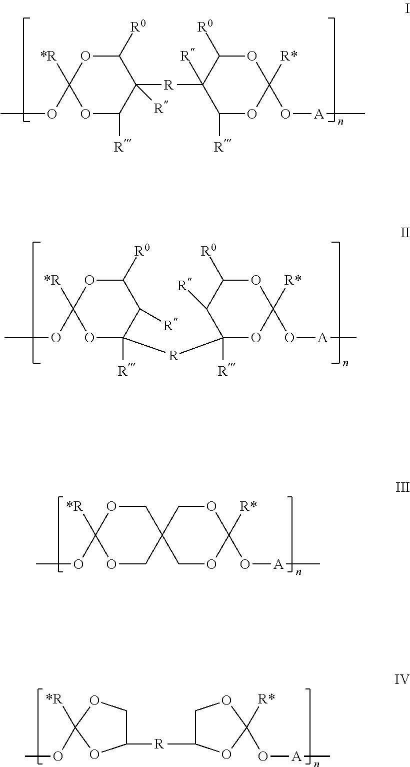

Polyorthoesters having a higher mole percentage of the “α-hydroxy acid containing” units will have a higher rate of bioerodibility. In one variation, the polyorthoesters are those in which the mole percentage of the “α-hydroxy acid containing” units is at least 0.01 mole percent, in the range of about 0.01 to about 50 mole percent, from about 0.05 to about 30 mole percent, for example from about 0.1 to about 25 mole percent, especially from about 1 to about 20 mole percent. The mole percentage of the “α-hydroxy acid containing” units appropriate to achieve the desired composition will vary from formulation to formulation.

In another variation, the polyorthoesters are those where: n is an integer of 5 to 1000; the polyorthoester has a molecular weight of 1000 to 20,000, 1000 to 10,000, or 1000 to 8000; R⁵ is hydrogen or methyl;

R⁶ is:

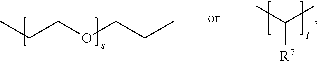

where s is an integer of 0 to 10, especially 1 to 4; t is an integer of 2 to 30, especially 2 to 10; and R⁷ is hydrogen or methyl;

R³ is:

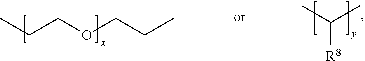

where x is an integer of 0 to 10, especially 1 to 4, preferably selected from 1, 2, 3, and 4; y is an integer of 2 to 30, or 2 to 10, particularly selected from 2, 3, 4, 5, 6, 7, 8, 9 and 10; and R⁸ is hydrogen or methyl;

R⁴ is selected from a residue of an aliphatic diol of 2 to 20 carbon atoms (e.g., selected from 2, 3, 4, 5, 6, 7, 8, 9, 10, 11, 12, 13, 14, 15, 16, 17, 18, 19, and 20 carbon atoms), or 2 to 10 carbon atoms, interrupted by one or two amide, imide, urea or urethane groups;

the proportion of units in which A is R¹ is about 0.01-50 mol %, or 0.05-30 mol %, or 0.1-25 mol %. Illustrative mole percentages include 10, 15, 20 and 25 mole percent of subunits in the polyorthoester in which A is R¹. In one embodiment, the mole percent is 20.

Additionally, in one or more embodiments, the proportion of subunits in which A is R2 is less than 20%, or less than 10%, especially less than 5%, and the proportion of units in which A is R⁴ is less than 20%, less than 10%, or less than 5%.

One illustrative polyorthoester is prepared from DETOSU:TEG:TEG-diGL, at a molar ratio of 90:80:20.

Methods of manufacturing the polyorthoesters are well known in the art.

### B. Excipients

Excipients for use in the compositions and delivery systems are pharmaceutically acceptable and polyorthoester-compatible materials. They are liquid at room temperature, and are readily miscible with the polyorthoesters.

Suitable excipients include poly(ethylene glycol) ether derivatives having a molecular weight of between 200 and 4,000, such as poly(ethylene glycol) mono- or di-alkyl ethers, preferably poly(ethylene glycol)monomethyl ether 550 or poly(ethylene glycol)dimethyl ether 250; poly(ethylene glycol)copolymers having a molecular weight of between 400 and 4,000 such as poly(ethylene glycol-co-polypropylene glycol); propylene glycol mono- or di-esters of a C₂₋₁₉ aliphatic carboxylic acid or a mixture of such acids, such as propylene glycol dicaprylate or dicaprate; mono-, di- or tri-glycerides of a C₂₋₁₉ aliphatic carboxylic acid or a mixture of such acids, such as glyceryl caprylate, glyceryl caprate, glyceryl caprylate/caprate, glyceryl caprylate/caprate/laurate, glycofurol and similar ethoxylated tetrahydrofurfuryl alcohols and their C₁₋₄ alkyl ethers and C₂₋₁₉ aliphatic carboxylic acid esters; and biocompatible oils such as sunflower oil, sesame oil and other non- or partially-hydrogenated vegetable oils.

In some embodiments, the excipients are aprotic solvents, and can be either water miscible, partially water miscible, or poorly water miscible, depending on the desired release profile for a given active agent and the solubility of the active agent in the polyorthoester polymer and polymer/solvent combination. It is also desired that the solvent be non-toxic. In one embodiment the solvent is selected such that it will quickly leave the composition after coming into contact with an aqueous environment, e.g. body fluids. In another embodiment, the solvent is selected such that, at least, some of the solvent will remain in the composition after coming into contact with body fluids.

In some embodiments a composition is comprised of a drug dissolved in a polymer/hydrophilic (water miscible) solvent combination, and the drug may be encapsulated or entrapped in the polymer matrix as the hydrophilic solvent dissolves or dissipates from the composition and into the body fluid. In other embodiments, a composition is comprised of a lipophilic (poorly water miscible) solvent, and the dissolution or diffusion of the lipophilic solvent into surrounding aqueous tissue fluid will be relatively slow with a resultant slower increase in viscosity of the administered composition. However, a lipophilic solvent, by its own nature, may slow the release of active agent incorporated into the composition until the solvent has dissipated, leaving the polymer at the site of delivery with the entrapped active agent. By adjusting the hydrophilicity/lipophilicity character of the polymer and/or the solvent, the release of the active agent can be controlled to provide a low initial burst and sustained release of both hydrophilic and lipophilic active agents. In addition, the solubility of a hydrophilic or lipophilic active agent can be controlled to provide either solutions or dispersions of the active agent in the liquid polymer/solvent compositions.

Suitable hydrophilic (water miscible) biocompatible organic solvents that may be used have, in one embodiment, water solubility greater than 10% by weight of the solvent in water. Examples of hydrophilic biocompatible organic solvents include amides such as N-methyl-2-pyrrolidone (NMP), 2-pyrrolidone, N-ethyl-2-pyrrolidone, N-cycylohexyl-2-pyrrolidone, dimethyl acetamide, and dimethyl formamide; esters of monobasic acids such as methyl lactate, ethyl lactate, and methyl acetate; sulfoxides such as dimethyl sulfoxide and decylmethylsulfoxide; lactones such as e-caprolactone and butyrolactone; ketones such as acetone and methyl ethyl ketone; and ethers such as dimethyl isosorbide and tetrahydrofuran.

Suitable lipophilic biocompatible organic solvents that may be used in the compositions and delivery systems described herein have, in one embodiment, a water solubility less than 10% by weight of the solvent in water. Examples of lipophilic biocompatible organic solvents include esters of mono-, di-, and tricarboxylic acids such as ethyl acetate, ethyl oleate and isopropyl myristate; and esters of aromatic acids such as benzyl benzoate.

Combinations of different hydrophilic solvents can be used to obtain higher or lower levels of solubility of the liquid polymer and bioactive agent in the resultant solution. A combination of organic solvents can also be used to control the rate of release of an active agent by controlling the rate at which the solvent dissolves or dissipates when the liquid polymer/solvent/active agent composition is placed in the body. Similarly, combinations of different lipophilic solvents can also be used to control the solubility of the liquid polymer and active agent in the solvent and the release of the active agent in the body. In other embodiments, combinations of hydrophilic and lipophilic solvents can be used to obtain the optimum solvent characteristics for a delivery system. Examples include a combination of N-methylpyrrolidone and isopropyl myristate which provides a more hydrophobic solvent than N-methylpyrrolidone alone, and a combination of N-methylpyrrolidone and another more soluble organic solvent, to provide a more hydrophilic solvent combination than N-methylpyrrolidone alone.

The organic solvent is typically added to the compositions in an amount ranging from about 10 percent to about 70 percent by weight, relative to the total weight of the composition. The solvent may be present in the composition in an amount ranging from about 20 percent to about 50 percent by weight. In other embodiments, the solvent may be present in the composition in an amount ranging from about 10-60 wt %, 15-60 wt %, 15-50 wt %, 20-60 wt %, 25-50 wt %, 30-70 wt %, 30-60 wt %, 30-50 wt %, 35-70 wt %, 35-60 wt % or 35-50 wt %. The concentration of solvent allows for the level of polymer in the composition to range from about 30 percent to about 90 percent by weight, or from about 50 percent to about 80 percent by weight relative to the overall composition.

In other embodiments, the compositions comprise between about 10 percent by weight to about 70 percent by weight solvent, relative to the combined weight of the polymer and solvent in the composition, or the compositions may comprise between about 20-50 percent by weight solvent, relative to the combined weight of the polymer and solvent in the composition. In other embodiments, the solvent may be present in the composition in an amount, relative to the combined amount of polymer and solvent in the composition, ranging from about 10-60 wt %, 15-60 wt %, 15-50 wt %, 20-60 wt %, 25-50 wt %, 30-70 wt %, 30-60 wt %, 30-50 wt %, 35-70 wt %, 35-60 wt % or 35-50 wt %. The concentration of solvent may allow for the level of polymer in the composition to range from about 30 percent to about 90 percent by weight, or from about 50 percent to about 80 percent by weight relative to weight of the polymer and solvent in the composition.

The polymer/solvent concentrations permit the liquid polymer/solvent compositions to be easily injected with standard syringes and small gauge needles (e.g., about 18-26 gauge) unlike liquid polymer formulations previously described, for example, which in some embodiments, unlike the present compositions, require the addition of a particulate material to achieve an acceptable viscosity for injection with a syringe and needle. The compositions of the invention can be administered into the body of a human subject or animal such as a dog, cat, horse, etc.

The rate of release of the active agent (e.g., drug) can be controlled by the composition of the biodegradable polymer and/or by the hydrophilicity or lipophilicity of the organic solvent that is used. The composition of the liquid polymer (i.e., the type of monomer used or the ratio of monomers for copolymers or terpolymers, the end groups on the polymer chains, and the molecular weight of the polymer) will determine the hydrophilicity or lipophilicity of the liquid polymer material as well as the degradation time of the liquid polymer depot. More hydrophilic liquid polymers (e.g., polyorthoesters wherein the diol monomer is hydrophilic, e.g., triethylene glycol, tetraethylene glycol, or polyethylene glycol and the like) and/or more hydrophilic solvents (e.g., N-methyl-2-pyrrolidone) can be used for active agents in applications where faster release rates and shorter durations of release (e.g., about 1-3 days) are needed. For slower releasing active agents and where longer durations of release for prolonged delivery (e.g., about 7-90 days) are desired, more hydrophobic and slower degrading liquid polymers (polyorthoesters wherein the diol monomer is hydrophobic, e.g., 1-6 hexanediol, 1-10 decanediol, or 1-12 dodecandiol and the like) and/or more lipophilic solvents (e.g., isopropyl myristate) can be used to advantage. For even slower rates and longer durations of release of an active agent, the active agent itself can be made more water-insoluble by utilizing active agents, for example, in the form of lipophilic salts, drug complexes, and/or prodrug esters, amides or ethers. Thus, various forms of the drug can be used as needed. The composition includes the active agent in an amount effective to provide the desired therapeutic effect over the release period. The concentration range of the active agent in the composition will vary, for example, according to the active agent, the formulation and the rate of release from the depot, and can range, for example, from about 0.1% to about 30% by weight. The liquid composition releases an effective amount of the bioactive agent by diffusion or dissolution from the composition as it biodegrades in the body.

While the singular form is used to describe the polyorthoester and solvent in this application, it is understood that more than one polyorthoester and/or more than one solvent selected from the groups described above may be used in the delivery system. It is also understood that while not required, other pharmaceutically acceptable inert agents such as coloring agents and preservatives may also be incorporated into the composition.

In some embodiments, when a polyorthoester is present in the pharmaceutical composition, the excipients are pharmaceutically acceptable and polyorthoester-compatible materials. In one embodiment, the excipients are liquid at room temperature, and are readily miscible with the polyorthoesters.

The compositions described herein are syringable or injectable, meaning that they can be dispensed from a conventional tube of the kind well known for topical or ophthalmic formulations, from a needleless syringe, or from a syringe with a 16 gauge or smaller needle (such as 16-25 gauge), and injected subcutaneously, intradermally or intramuscularly. The formulations may be applied using various methods known in the art, including by syringe, injectable or tube dispenser.

### C. Active Agents

An “active agent” or “active ingredient” refers to any compound or mixture of compounds which produces a beneficial or useful result. Active agents are distinguishable from such components as vehicles, carriers, diluents, lubricants, binders and other formulating aids, and encapsulating or otherwise protective components. Examples of active agents are pharmaceutical, agricultural or cosmetic agents. Suitable pharmaceutical agents include locally or systemically acting pharmaceutically active agents which may be administered to a subject by topical or intralesional application (including, for example, applying to abraded skin, lacerations, puncture wounds, etc . . . , as well as into surgical incisions) or by injection, such as subcutaneous, intradermal, intramuscular, intraocular or intra-articular injection. Suitable pharmaceutical agents include polysaccharides, DNA and other polynucleotides, antisense oligonucleotides, antigens, antibodies, vaccines, vitamins, enzymes, proteins, naturally occurring or bioengineered substances, and the like, anti-infectives (including antibiotics, antivirals, fungicides, scabicides or pediculicides), antiseptics (e.g., benzalkonium chloride, benzethonium chloride, chlorhexidine gluconate, mafenide acetate, methylbenzethonium chloride, nitrofurazone, nitromersol and the like), steroids (e.g., estrogens, progestins, androgens, adrenocorticoids and the like), opioids (e.g. buprenorphine, butorphanol, dezocine, meptazinol, nalbuphine, oxymorphone and pentazocine), therapeutic polypeptides (e.g. insulin, erythropoietin, morphogenic proteins such as bone morphogenic protein, and the like), analgesics and anti-inflammatory agents (e.g., aspirin, ibuprofen, naproxen, ketorolac, COX-1 inhibitors, COX-2 inhibitors and the like), antipsychotic agents (for example, phenothiazines including chlorpromazine, triflupromazine, mesoridazine, piperacetazine and thioridazine; thioxanthenes including chlorprothixene and the like), antiangiogenic agents (e.g., combresiatin, contortrostatin, anti-VEGF and the like), anti-anxiety agents (for example, benzodiazepines including diazepam, alprazolam, clonazepam, oxazepam; and barbiturates), anti-depressants (including tricyclic antidepressants and monoamine oxidase inhibitors including imipramine, amitriptyline, doxepin, nortriptyline, amoxapine, tranylcypromine, phenelzine and the like), stimulants (for example, methylphenidate, doxapram, nikethamide and the like), narcotics (for example, buprenorphine, morphine, meperidine, codeine and the like), analgesic-antipyretics and anti-inflammatory agents (for example, aspirin, ibuprofen, naproxen and the like), local anesthetics (e.g., the amide- or anilide-type local anesthetics such as bupivacaine, dibucaine, mepivacaine, procaine, lidocaine, tetracaine and the like), fertility control agents, chemotherapeutic and anti-neoplastic agents (for example, mechlorethamine, cyclophosphamide, 5-fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen and the like), cardiovascular and anti-hypertensive agents (for example, procainamide, amyl nitrite, nitroglycerin, propranolol, metoprolol, prazosin, phentolamine, trimethaphan, captopril, enalapril and the like), drugs for the therapy of pulmonary disorders, anti-epilepsy agents (for example, phenytoin, ethotoin and the like), anti-hidrotics, keratoplastic agents, pigmentation agents or emollients, antiemetic agents (such as ondansetron, granisetron, tropisetron, metoclopramide, domperidone, scopolamine and the like). The composition of the present application may also be applied to other locally acting active agents, such as astringents, antiperspirants, irritants, rubefacients, vesicants, sclerosing agents, caustics, escharotics, keratolytic agents, sunscreens and a variety of dermatologics including hypopigmenting and antipruritic agents. The term “active agents” further includes biocides such as fungicides, pesticides and herbicides, plant growth promoters or inhibitors, preservatives, disinfectants, air purifiers and nutrients. Pro-drugs and pharmaceutically acceptable salts of the active agents are included within the scope of the present application.

In one embodiment, the active agent is an antiemetic agent. Exemplary amtiemetic agents include 5-HT₃ antagonists, dopamine antagonists, anticholinergic agents, GABA_{B} receptor agonists, NK₁ receptor antagonists, and GABA_{A}alpha₂ and/or alpha₃ receptor agonists. In one embodiment the active agent is a 5-HT₃ antagonist selected from the group consisting of ondansetron, granisetron and tropisetron.

The active agent or agents can be dissolved or dispersed into the composition comprising a polyorthoester and a biocompatible solvent. The concentration of the active agent in the composition may vary from about 1 wt % to 20 wt %, 1 wt % to 10 wt %, 10 wt % to 20 wt %, 2 wt % to 5 wt %, 10 wt % to 15%, or 15 wt % to 20 wt % and may be 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, 10 wt %, 11 wt %, 11.1 wt %, 11.2 wt %, 11.3 wt %, 11.4 wt %, 11.5 wt %, 11.6 wt %, 11.7 wt %, 11.8 wt %, 11.9 wt %, 12 wt %, 12.1 wt %, 12.2 wt %, 12.3 wt %, 12.4 wt %, 12.5 wt %, 12.6 wt %, 12.7 wt %, 12.8 wt %, 12.9 wt %, 13 wt %, 13.1 wt %, 13.2 wt %, 13.3 wt %, 13.4 wt %, 13.5 wt %, 13.6 wt %, 13.7 wt %, 13.8 wt %, 13.9 wt %, 14 wt %, 14.1 wt %, 14.2 wt %, 14.3 wt %, 14.4 wt %, 14.5 wt %, 14.6 wt %, 14.7 wt %, 14.8 wt %, 14.9 wt %, 15 wt %, 15 wt %, 15.1 wt %, 15.2 wt %, 15.3 wt %, 15.4 wt %, 5.5 wt %, 15.6 wt %, 15.7 wt %, 15.8 wt %, 15.9 wt %, 16 wt %, 16.1 wt %, 16.2 wt %, 16.3 wt %, 16.4 wt %, 16.5 wt %, 16.6 wt %, 16.7 wt %, 16.8 wt %, 16.9 wt %, 17 wt %, 17.1 wt %, 17.2 wt %, 17.3 wt %, 17.4 wt %, 17.5 wt %, 17.6 wt %, 17.7 wt %, 17.8 wt %, 17.9 wt %, 18 wt %, 18.1 wt %, 18.2 wt %, 18.3 wt %, 18.4 wt %, 18.5 wt %, 18.6 wt %, 18.7 wt %, 18.8 wt %, 18.9 wt %, 19 wt %, 19.1 wt %, 19.2 wt %, 19.3 wt %, 19.4 wt %, 19.5 wt %, 19.6 wt %, 19.7 wt %, 19.8 wt %, 19.9 wt %, 20 wt %.

The compositions may comprise a second active agent. In one embodiment, the first and second antiemetic agents are included in the composition. In one variation, the second antiemetic agent is selected from the group consisting of alpha-2 adrenoreceptor agonists, a dopamine antagonist, an anticholinergic agent, a GABA_{B} receptor agonist, an NK₁ receptor antagonist, and a GABA_{A}alpha₂ and/or alpha₃ receptor agonist. In another variation, the alpha-2 adrenoreceptor agonists is selected from the group consisting of clonidine, apraclonidine, para-aminoclonidine, brimonidine, naphazoline, oxymetazoline, tetrahydrozoline, tramazoline, detomidine, medetomidine, dexmedetomidine, B-HT 920, B-HIT 933, xylazine, rilmenidine, guanabenz, guanfacine, labetalol, phenylephrine, mephentermine, metaraminol, methoxamine and xylazine.

### III. Heat-Generating Component

The heat-generating component for use in the claimed method, systems and kits is based, in one embodiment, on a thermochemical composition which can be activated at the time of use. A variety of thermochemical compositions are suitable and examples are set forth below. In one embodiment, the self-generating heat source is comprised of two or more chemical components that are physically separated prior to use by a barrier. Activation of the heat source is achieved by rupture of the physical barrier to permit combination of separate chemical components. In another embodiment, an exothermic solid composition e.g., a metal powder, brings about an exothermic reaction in the presence of air or oxygen. Examples of these embodiments, and other possible self-generating heat sources will now be described.

In one embodiment, the exothermic heat-generating component comprises a particulate solid. It could be present, for example, as granules, pellets or slugs. In one embodiment, the exothermic heat-generating component comprises iron. The heat-generating material may further comprise carbon, metal salts and water. In a preferred embodiment, the heat source comprises iron powder as a main ingredient as described in U.S. Pat. Nos. 5,046,479 and 5,918,590, and U.S. Patent Application Pub. No. 2007/0034202, each of which is incorporated herein by reference in its entirety. Certain exothermic compositions based on iron oxidation chemistry are known in applications to different exothermic devices (e.g., U.S. Pat. Nos. 4,366,804; 5,046,479; 6,099,556; 5,984,995; and 5,042,455; each of which is incorporated herein by reference in its entirety.

In this embodiment, the heat-generating component is placed within an oxygen permeable containment or enclosure. The containment may be segmented into small pockets to keep the heat-generating component evenly distributed throughout the containment. Alternately, the heat-generating component may be free flowing within the containment. The permeable layer may comprise, for example, a non-woven material or alternately a microporous film. The permeable layer may comprise one or more of the surfaces of the containment.

As an example, the above-described object of the present invention can be attained by a disposable body warmer wherein the air permeability per unit time in an air-permeable surface of an enclosure is limited to 5000 to 10000 sec/100 cc so as to bring about a reduction in the pressure accompanying oxidative heat generation of the heat generating agent packed in the enclosure, wherein the heat generating agent comprises iron powder as a main ingredient and, mixed therewith, 9% to 11% by weight of a water-retaining agent, 18% to 22% by weight of water, a heat generation promoter and salt and packed in a flat form having a thickness of 2 mm to 5 mm in the flat enclosure. A nontransferable self-adhesive layer is attached over or around the permeable layer enclosure to allow attachment of a sealing layer. The sealing layer is removed to allow air ingress into the enclosure to initiate the oxidation reaction and generate heat.

The limitation of the air permeability of the air-permeable surface of the enclosure to 5000 sec/100 cc or less causes the oxidative heat generation of the heat generating agent mainly composed of iron powder placed in the enclosure to reduce the pressure within the enclosure. That is, the amount of supply of oxygen (amount of air) is limited to a value less than that necessary for oxidation of that agent so that the above-described enclosure can be maintained in a compression-flattened state under atmospheric pressure during the oxidative heat generation. This compression-flattened enclosure prevents the uneven distribution of the heat generating agent within the enclosure and equalizes the temperature distribution.

The composition of the heat generating composition may be adjusted to provide the desired temperature within the desired time period. Such variations are well known in the art. For example, limitation of the amount of water to 18% by weight or more enables an exothermic reaction utilizing iron powder to properly proceed. On the other hand, limitation of the maximum amount thereof to 22% by weight enables set up of a proper heat generation state upon exposure of the iron powder to air or oxygen, i.e., enables excellent initiation of the heat generation. This prevents the heat generating agent from being unevenly distributed by virtue of proper reduction in the pressure within the enclosure from the initiation of use of the warmer and brings about a favorable thermal effect.

Limitation of the thickness of the heat generating agent packed in the above-described enclosure to 2 mm or more contributes to the setup of a heat generation state or temperature distribution favorable for warming the housing or device for the substance, while limitation of the maximum thickness thereof to 5 mm or less restricts the load per unit area of the agent mainly composed of iron powder to thereby aid the prevention of uneven distribution of the agent.

The exothermic device according to the present invention may be formed by injecting or inserting the exothermic composition into an enclosure formed of a film and having at least one gas-permeable surface, and sealing open sides thereof. The type of sealing may be side sealing, two-side sealing, three-side sealing, envelope type or mid-joint sealing. The pouch is often sealed by heat sealing. However, where opposite films or sheets are formed of a material not fit for heat sealing, a hot melt type adhesive or hot melt adhesive film, or a paste, may be interposed between the opposite plastic films.

At least one surface of the enclosure may be made gas-permeable, for example, by punching a gastight pouch to form numerous pores therein. To simplify a pouch manufacturing process, one or both surfaces of the pouch may be formed of a gas-permeable plastic film obtained by a drawing process, woven or nonwoven fabric (including paper), or a combination thereof (hereinafter referred to as gas-permeable film).

The material for the gas-permeable film is not limited to any particular material, but may be a known material conventionally used for a pouch which encloses an exothermic composition. Usable materials include, for example, paper, polyethylene, polypropylene, polyamide, polyester, polyvinyl chloride, polyvinylidene chloride, polyurethane, polystyrene, saponified ethylene-vinyle acetate copolymer, ethylene-vinyle acetate copolymer, natural rubber, reclaimed rubber and synthetic rubber.

The gas permeability of the gas-permeable film influences control of the exothermic temperature and heating time of the exothermic device. Conventionally, it is preferred to control the gas permeability of the film by means of water-vapor permeability to effect a particularly strict temperature control of the exothermic device in order to obtain an effective heating effect and to secure safety by avoiding a low-temperature burn.

In an alternative embodiment, the heat source comprises a supercoolable aqueous salt solution. Examples of such exothermic thermochemical compositions can include, but are not limited to: sodium thiosulfate liquid and borax solid, sodium acetate liquid and sodium acetate solid, magnesium sulfate compositions, and the like. Detailed descriptions of these thermochemical compositions can be found, for example, in U.S. Pat. Nos. 5,143,048 and 5,295,964 and U.S. Patent Application Pub. Nos. 2005/0228466 and 2012/0193347, each of which is incorporated by reference in its entirety.

In one embodiment, the heat source contains a single liquid thermochemical composition that produces exothermic temperatures alongside its crystallization, and a physical activator associated within the composition. Physical activators can be can be readily and visibly located by the individual and then activated at time of use. This category of activator has been referred to in a variety of ways in the art, for example as “clickers” and initiators, and are herein referred to as triggers. By virtue of their structure, the trigger can be a compact, flat, relatively small structure, the rubbing, bending or flexing of which initiates crystallization of the thermochemical composition. The crystallization in turn is associated with exothermic warming temperatures. The use of a trigger facilitates activation of the thermochemical composition of the device.

Physical activators that can be used in accordance with the invention can include triggers in the form of particles adhered to a substrate surface or flexible metallic discs. Examples of such triggers include, but are not limited to, aluminum oxide particle or grit surface materials and flexible stainless steel triggers. Flexible stainless steel triggers can comprise a flat stainless steel disc containing one or more ridges, slots or openings there through.

For exothermic thermochemical liquid composition and physical activator systems, suitable exothermic thermochemical liquids include, but are not limited to, sodium acetate trihydrate. In a preferred embodiment, the thermochemical composition comprises a mixture of sodium acetate trihydrate present in an amount of about 73% of the total liquid volume and water present in an amount of about 27% of the total liquid volume; and the physical activator comprises a trigger comprising an aluminum oxide grit or comprising a flexible stainless steel trigger.

Unlike the containment for the solid particular exothermic compositions (e.g., iron powder as described above), the exothermic liquid compositions are contained in an impermeable flexible container which is not affected by the solution.

The heat source of the present disclosure comprises, in one embodiment, a flexible polymeric containment for the thermochemical composition, such as that described in U.S. Pat. No. 5,143,048 and U.S. Patent Application Pub. Nos. 2005/0228466 and 2012/0193347, each of which is incorporated herein by reference in its entirety, wherein the containment is conformable to the housing or device containing the delivery system substance, e.g., syringe or vial. The housing or device may be plastic, glass or other suitable material. The containment may have an overall flat or planar configuration. A variety of containment shapes and configurations can be used in accordance with the present disclosure. In one embodiment, the containment comprises a relatively flat rectangular shape. When this configuration is employed, the housing is placed along one edge of the containment, then rolled such that the surface of the containment adheres to the housing until the housing is completely surrounded by the containment. In one embodiment, the containment may be manipulated in order to activate the exothermic thermochemical liquid composition prior to wrapping the containment around the housing. In another embodiment, the exothermic composition is activated as the containment is wrapped around the housing for the delivery system.

Alternatively, the containment may be a tubular sleeve with a lumen which is sized accordingly to receive the housing or device for the delivery system substance. The sleeve may be open at one end or at both ends to receive the housing or device. The sleeve may be placed within the heating source housing or may be on one of the surfaces of the heating source housing.

The containment may be constructed so as to be foldable about the housing or device. The containment may include means to secure the heat source about the housing or device. Such means may include adhesive tape, hook and loop fasteners, mechanical snaps or other securement means.

The flexible polymeric containment can be constructed from a variety of flexible polymeric film. Suitable film materials that can be used include polyester, polypropylene, polyethylene, nylon, ethyl vinyl acetate (EVA), and combinations thereof. The flexible polymeric containment material can be single layered or multilayered. A flexible polymeric film material for the invention is a barrier film comprising layers of nylon and polyethylene. An alternative flexible polymeric film material for the invention is a thermal barrier film comprising an interior layer composed of a blend of linear low density polyethylene (LDPE) and ethyl vinyl acetate (EVA), and polyester layer coated with aluminum oxide.

The flexible polymeric containment may comprise a viewing window, which is a transparent portion which allows a user to observe the warming delivery system during the warming period and/or to observe the level of delivery system remaining in the housing if, for example, the housing is a syringe used to inject the delivery system into a subject.

The containment may have an adhesive layer. The adhesive layer is not limited to any particular type as long as it can be affixed directly to the housing for the delivery system.

The containment may comprise a temperature sensitive indicating label to indicate the temperature of the heat source. The temperature sensitive label may be a one-way indicating or reversible indicator. The temperature sensitive may be comprised of a liquid crystal type temperature indicator. The containment system may alternately contain labeling which is comprised of temperature sensitive ink. The ink may be one-way indicating or reversible.

The adhesive layer may be a layer formed of an adhesive, or a layer formed of a compress to produce a hot compress effect regardless of presence of moisture. The adhesive is not limited as long as it is a high polymer material having adhesive property. The invention may use various types of rubber adhesives and acrylic adhesives widely used to date as adhesives of application pads. A hot-melt type adhesive may also be used. The adhesive may be a combination of two or more adhesives. (See disclosure for adhesives in U.S. Pat. No. 6,099,556, incorporated herein by reference.)

A release paper is adhered to the adhesive layer and the containment is put in an air-impermeable packaging bag, whereupon hermetic sealing of the periphery of the packaging bag is conducted. Removal of the release paper allows exposure of the exothermic material within the containment and activation of the exothermic reaction. Materials of the packaging bag are not limited insofar as they are air tight. Laminated films may be employed. The packaging bag made be comprised of films made of, for example, OPP (oriented polypropylene), CPP (casting polypropylene), films of nylons, polyesters and polypropylenes having a moisture barrier coating of polyvinylidene chloride thereon; aluminum foil; plastic films having an aluminum deposition layer, and the like.

### IV. Methods of Treatment

In another aspect, the compositions and systems described herein are for treatment of a subject, and the composition or system is administered via injection to a subject in need.

In one embodiment, the compositions are for use in a method for the treatment of emesis induced by a chemotherapeutic agent, by radiation-induced nausea and vomiting, and/or by post-operative induced nausea and vomiting in a patient. The treatment includes administering to the patient a composition comprising an anti-emetic, such as a 5-HT₃ antagonist according to the method described herein where the composition is contacted with a self-generating heat source prior to administration to the patient.

More generally, the compositions and systems are administered to a subject (e.g., patient) in need of a treatment or prevention of a condition, an effective amount of the flowable composition described herein. The compositions provide the advantages of liquid application to form medical or surgical devices and/or delivery systems for active agents (e.g., drugs). The present liquid polymer/solvent compositions also allow the use of smaller gauge needles compared to other liquid polymer systems made without a solvent. The solvents used in the present compositions allow an active agent to also be administered as a solution in contrast to liquid polymer systems made without solvents. The use of liquid biodegradable polymers in the present system also allows the rate of release of an active agent and degradation of the flowable composition to be varied over a wide range in contrast to the nonpolymeric flowable compositions.

### V. Examples

The following examples are illustrative in nature and are in no way intended to be limiting.

### Example 1

### Measurement of Viscosity

Compositions, 2 to 5 grams of each, of a polyorthoester (POE) of formula III, granisetron base and varying amounts of dimethyl sulfoxide or N-methyl pyrrolidone are prepared by dissolving the appropriate amount of granisetron base into each solvent at approximately 80° C. The drug solutions are then mixed with the appropriate amount of polymer at an elevated temperature, until homogeneous, to form compositions with 10%, 20%, and 30% solvent and 2% granisetron base. Aliquots of the homogeneous solutions are then placed into vials or syringes. A heat pad is activated and adhered to the container, and the container is inverted over a period of time, for example for about 30 seconds, 1 minute, or about 2, 3, 5, 8 or 10 minutes. Viscosity of the compositions is then measured using a Brookfield cone and plate viscometer. The viscosity measurements are performed at 37° C.

### Example 2

### Method of Administration

A syringe comprising a composition of a polyorthoester (POE) of formula III, granisetron base and a solvent is prepared by loading into the barrel of the syringe the composition. The syringe is placed in contact with a self-generating, portable heat source contained in a flexible housing, so that the flexible housing can be wrapped about the syringe. The heat source is held in contact with the syringe for between 30 seconds-1 minute. Then the needle of the syringe is inserted subcutaneously into the patient to deliver the warmed composition.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for administering a polymer-based delivery system to a subject, comprising:
contacting a housing comprising a polymer-based delivery system with a heat-generating component for a period of time, wherein the polymer-based delivery system comprises (i) a polyorthoester of formula III: 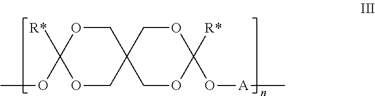
where
R* is a C₁₋₄ alkyl;
n is an integer of at least 5;
A is R¹ or R³, where
R¹ is: 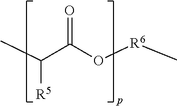
p is an integer of 1 to 20;
R⁵ is hydrogen;
R⁶ is 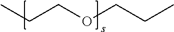
s is an integer from 1 to 4;
R³ is 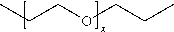
x is an integer from 1 to 4;
in which 20 mol percent of the A units are of the formula R¹;
wherein the polyorthoester is prepared from 3,9-di(ethylidene)-2,4,8,10-tetraoxaspiro[5.5]undecane (DETOSU), triethylene glycol (TEG) and triethylene glycol diglycolide (TEG-diGL);
(ii) a pharmaceutically acceptable liquid excipient which is a polyethylene glycol ether having a molecular weight between 200 and 4000; and
iii) from about 1 to 5 weight percent granisetron;
and
injecting the delivery system into the subject.

2. The method of claim 1, further comprising activating the heat-generating component before the contacting.

3. The method of claim 1, further comprising inverting or agitating the housing during the period of time.

4. The method of claim 1, wherein the heat-generating component comprises an exothermic thermochemical composition.

5. The method of claim 4, wherein the exothermic composition comprises a supercoolable aqueous salt solution.

6. The method of claim 1, wherein the heat-generating component comprises a solid exothermic thermochemical composition.

7. The method of claim 6, wherein the solid exothermic composition is an iron powder, wherein an exothermic reaction is activated upon exposure of the exothermic composition to air.

8. The method of claim 6, further comprising activating the heat-generating component before the contacting.

9. (canceled)

10. The method of claim 1, wherein the period of time ranges from about 10 seconds to about 10 minutes.

11. The method of claim 1, wherein the viscosity of the delivery system decreases to less than 10,000 centipoise when measured at a temperature of about 25° C.

12. The method of claim 1, wherein the delivery system reaches a temperature of about 35° C. to 45° C. at the end of the period of time.

13. The method of claim 1, wherein the delivery system is injected into the subject within about 5 seconds to 2 minutes after the end of the period of time.

14. The method of claim 1, wherein the injecting is subcutaneous, intradermal or intramuscular.

15. A kit comprising a housing in which a polymeric-based delivery system is contained and a container comprising a heat-generating component.

16. The kit of claim 15, wherein the container is a flexible pad comprising a first surface and a second surface, wherein the heat-generating component is encased between the first and second surface.

17. The kit of claim 16, wherein the first surface comprises an adhesive.

18. The kit of claim 17, wherein the container is tubular in shape and comprises a lumen.

19. The kit of claim 15, wherein the housing is a syringe, a bottle or a vial.
